(19) United States
(12) Patent Application Publication
(10) Pub. No.: US 2023/0119928 A1
(43) Pub. Date: Apr. 20, 2023

(54) PLATELET LYSATE FOAM FOR CELL CULTURE, CELL THERAPY AND TISSULAR REGENERATION AND METHOD FOR OBATINING SAME

(71) Applicants: UNIVERSITE PAUL SABATIER TOULOUSE III, Toulouse (FR); CENTRE HOSPITALIER UNIVERSITAIRE DE TOULOUSE, Toulouse Cedex 9 (FR)

(72) Inventors: Sophie CAZALBOU, Toulouse (FR); Thibault CANCEILL, Toulouse (FR)

(21) Appl. No.: 17/906,989

(22) PCT Filed: Mar. 15, 2021
(86) PCT No.: PCT/FR2021/050427

§ 371 Date: Sep. 22, 2022

(30) Foreign Application Priority Data
Mar. 23, 2020 (FR) 20 02800

(51) Int. Cl. A61K 35/19; A61K 9/12; A61K 38/18; A61K 38/30; A61K 33/06; A61K 31/195; A61K 47/42; A61L 27/56; A61L 27/36

(52) U.S. Cl. CPC A61K 35/19; A61K 9/122; A61K 38/1841; A61K 38/1808; A61K 38/1858; A61K 38/30; A61K 38/1866; A61K 38/1825; A61K 33/06; A61K 31/195; A61K 47/42; A61L 27/56; A61L 27/3616; A61L 2430/02; A61L 2430/12

(57) ABSTRACT

The present invention relates to a platelet lysate foam obtained from blood derivative (allogenic or autologous) which retains the biological properties of the platelet lysate and has optimal properties, in particular mechanical but also storage, which allow sale thereof and make handling thereof easier.

The present invention also relates to the use of a platelet lysate foam for therapeutic purposes, cell culture and cell therapy.

The present invention also relates to a process for getting a platelet lysate foam by a process of drying in a supercritical CO₂ atmosphere.

### FIELD OF THE INVENTION

The present invention relates to the preparation of a biomaterial obtained by drying a platelet lysate (PL) hydrogel with supercritical CO₂.

### DESCRIPTION OF RELATED ART

Platelet lysate (PL) is a blood derivative rich in growth factors. It is routinely used for cell culture and routes exist for its possible use in human therapies. Platelet lysate obtained by simple destruction of the plasma membrane of platelets circulating in the blood currently presents new strategies for cell culture, healing and tissue regeneration.

In fact, the presence in platelet lysates of growth factors and cytokines such as VEGF, PDGF, EGF and TGF-β which are released during placement of the concentrate in the medium (thereby contributing to the growth of the tissues) constitutes a major argument for the “biomedical” use of platelet concentrates (Amable P R et al., Mesenchymal stromal cell proliferation, gene expression and protein production in human platelet-rich plasma-supplemented media. PloS One 2014; 9(8):e104662).

Hydrogels of platelet lysate have been proposed in the state-of-the-art. However, the presence of water in the platelet lysate and the gel does not allow good storage or good handling for in vivo implanting.

Gelling is a process which causes, within the solution, a solid phase to appear which organizes forming a continuous three-dimensional network which will trap the solvent.

The gel is therefore a thermodynamically stable solid-liquid biphase system made up of a double three-dimensional continuous interpenetrating network, one solid the other liquid.

Systems there are alternatives to hydrogels have been proposed, like foams. The foam is a gas dispersion in a condensed phase, in other words, it is a familiar system with complex behavior and ambiguous properties. For example, foams have a very low density, but can sometimes be perfectly stiff, even solid.

A foam with a mixture of fibrin and other substances such as thrombin, prothrombin, blood platelet extracts, protease inhibitors, antibiotics, for absorbing biochemical substances and substrates for accelerated hemostasis and an optimized biochemical control for closure of the wound can be given as an example. The foam is obtained by freeze-drying (U.S. Pat. No. 4,442,655). However, freeze-drying requires a step of freezing of the fiber network which, when it is poorly controlled, leads to bursting of the foam and makes it unusable. Further, freeze-drying, unless it is done in a clean room, does not allow manufacturing sterile biomaterials. Freeze-drying in a clean room additionally imposes a constraint and additional costs.

Fibrin foams and matrices with improved controlled delivery are also described in the document US 2013/183279. Bioactive factors such as growth factors are added before polymerization of the fibrin. These bioactive factors are therefore added and are not naturally present in the precursor composition.

### Technical Problem

It is thus necessary to conceive of a biomaterial of natural origin which can be handled easily, has good storage and which is also capable of inducing tissue regeneration in the host by the formation of a matrix which supports invasion, development, proliferation and activation of the cells of the receiver. It is also necessary to propose a process that is simple to implement and that serves to obtain a sterile biomaterial.

### BRIEF SUMMARY OF THE INVENTION

The present invention relates to a platelet lysate foam obtained from blood derivative (allogenic or autologous) which retains the biological properties of the platelet lysate and has optimal properties, in particular mechanical but also storage, which allow sale thereof and make handling thereof easier.

The platelet lysate foam according to the present invention may be used directly in the dry state thus allowing immediate penetration of cells, growth factors and biological fluids present on the placement site, or hydrated to recover the gelled form. It also allows slow and extended release of growth factors naturally present in the platelet lysate foam.

Because of the slow and extended release of the growth factors, the platelet lysate foam according to the invention advantageously supports the cell invasion, development and proliferation.

Thus, the platelet lysate foam according to the present invention is advantageously used for therapeutic purposes, cell culture and may be considered for cell therapy purposes.

The present invention also relates to a process for getting a platelet lysate foam by drying in a supercritical CO₂ atmosphere and a platelet lysate foam which could be obtained by this process.

### DETAILED DESCRIPTION OF THE INVENTION

The present invention also relates to a platelet lysate foam characterized in that it comprises TGF-beta, EGF, PDGF-AB, IGF-1, VEGF and bFGF within a polymerized fibrin matrix.

Platelet lysate foams according to the present invention are obtained directly from platelet lysates and advantageously retain the biological properties of the platelet lysates.

A foam is a gas dispersion in a condensed phase. In the domain of foams, two kinds are distinguished: foams, called moist, which contain a high liquid fraction by volume and which can be considered as dispersions of gas in a liquid, and the other, called dry, which contained very little liquid.

The foam according to the present invention is a dry foam.

Typically, the water concentration in the foam according to the invention is below 10% relative to the total weight of the foam, preferably below 7.5% and more preferably, the water concentration is below 5%.

Typically, the water concentration of the foam according to the invention is about 4.5%.

The water concentration may be measured by any technique known to the person skilled in the art. Typically, infrared balance or thermogravimetry is mentioned.

Platelet Lysate

Platelet lysate is understood to mean the product of lysis of platelets, meaning the product resulting after disintegration of the cellular membrane which leads to the release of molecules such as growth factors and cytokines normally contained inside the platelets.

Platelet lysate used for the manufacture of the foam may be obtained by purchasing pools of platelet lysate designed from blood samples from several donors or by direct design from collection from a patient. Blood, collected in citrated tubes, is centrifuged to separate the red globule, white globule and plasma phases. The isolation of the plasma concentrated in platelets then allows it to undergo cycles of freezing-thawing or sonication in order to destroy the platelet membranes and result in the platelet lysate. A leucodepletion phase is applicable for eliminating any residue of leukocytes in the solution.

Naturally Present

The growth factors present in the platelet lysate foam are growth factors naturally present in the platelet lysate (Fekete et al. “Platelet lysate from whole blood-derived pooled platelet concentrates and apheresis-derived platelet concentrates for the isolation and expansion of human bone marrow mesenchymal stromal cells: production process, content and identification of active components. 2012 May; 14(5):540-54. doi: 10.3109/14653249.2012.655420. Epub 2012 Feb. 2).

Thus, “naturally present” is understood to mean the fact of obtaining platelet lysate foams comprising growth factors present in the platelet lysate.

“Naturally present” is the opposite of “added.” In fact, growth factors present in the platelet lysate foam according to the present invention are present in the precursor composition, meaning in the platelet lysate used for getting the platelet lysate foam.

In fact, the process according to the invention advantageously serves to retain the elements in the platelet lysate which are precursors of the platelet lysate foam according to the invention.

The term naturally present is the opposite of the terms “additional,” “added” or any other synonym or even the term “added bioactive factor,” such as used in the state-of-the-art, for example in the application US 2013/0183279. In the state-of-the-art, “additional” or “added bioactive factor” designates a bioactive factor (for example a growth factor and/or a cytokine and/or bioactive ions) which is not present in the composition of the precursor, the fibrin formulation and/or the fibrin matrix, but which is added in the laboratory to the precursor composition and/or the fibrin formulation and/or matrix. These bioactive factors are therefore “artificially” incorporated in the formulation during formation of the foam.

Advantageously, the presence of human origin growth factors in natural quantities in the precursor is compatible with the mechanisms of human healing and tissue regeneration.

It is not necessary to add growth factors, unlike the biomaterial from the state-of-the-art, in order to get the biological properties of platelet lysates.

The platelet lysate contains between 110 and 150 pg/mL of β FGF (relative standard deviation: 8.09%), between 550 and 600 pg/mL of VEGF (relative standard deviation: 5.03%), between 25 and 29 ng/mL of PDGF-AB (relative standard deviation: 7.77%), between 70 and 75 mg/mL of TGF-beta (relative standard deviation: 4.34%), about 2 ng/mL of EGF (relative standard deviation: 6.02%), between 60 and 80 ng/mL of IGF-1 (according to the composition of platelet lysate LP 100 sold by MACOPHARMA).

For example, the platelet lysate that is the precursor of the platelet lysate foam according to the invention comprises about 2 ng/mL of EGF, 26.5 mg/mL of PDGF-AB, 72.5 ng/mL of IGF-1, 575 pg/mL of VEGF, 125 pg/mL of β FGF and 70 ng/mL of TGF-beta.

The concentrations of growth factors of the platelet lysate foams according to the present invention are proportional to the quantity of lysate used to create the foam. Advantageously, concentrations of growth factors in the final foam are not affected by the preparation process.

Typically, the concentration of TGF-beta in the foam according to the present invention is included between 1.84·10⁻³% by mass and 1.84·10⁻⁵% by mass, and is preferably about 7·10⁻⁷ g in 3.8·10⁻² g of foam or 1.84·10⁻⁴% by mass.

Typically, the concentration of EGF in the foam according to the present invention is included between 3.63·10⁻⁵% by mass and 3.63·10⁻⁷% by mass, and is preferably about 1.38·10⁻⁹ g in 3.38·10⁻² g of foam or 3.63·10⁻⁶% by mass.

Typically, the concentration of PDGF-AB in the foam according to the present invention is included between 4.79·10⁻⁴% by mass and 4.79·10⁻⁶% by mass, and is preferably about 1.82·10⁻⁸ g in 3.8·10⁻² g of foam or 4.79·10⁻⁵% by mass.

Typically, the concentration of IGF-1 in the foam according to the present invention is included between 1.31·10⁻³% by mass and 1.31·10⁻⁵% by mass, and is preferably about 4.99·10⁻⁸ g in 3.8·10⁻² g of foam or 1.31·10⁻⁴% by mass.

Typically, the concentration of VEGF in the foam according to the present invention is included between 1.04·10⁻⁵% by mass and 1.04·10⁻⁷% by mass, and is preferably about 3.95·10⁻¹⁰ g in 3.38·10⁻² g of foam or 1.04·10⁻⁶% by mass.

Typically, the concentration of bFGF in the foam according to the present invention is included between 2.26·10⁻⁶% by mass and 2.26·10⁻⁸% by mass, and is preferably about 8.6·10⁻¹¹ g in 3.8·10⁻² g of foam or 2.26·10⁻⁷% by mass.

According to an embodiment, the platelet lysate foam according to the present invention additionally comprises tranexamic acid and/or calcium.

According to an embodiment, the platelet lysate foam according to the present invention additionally comprises tranexamic acid and/or calcium, and/or chloride, and/or sodium.

Advantageously, the platelet lysate foam according to the present invention does not affect the activities of the growth factors and retains the properties of these growth factors along with the elements added to form the lysate hydrogel and in particular the elements preferably added are sodium, chloride, tranexamic acid and calcium. Thus, except for solvents, all the elements added to the formula for the hydrogel which after drying will form the foam are retained in the final dry material. These elements could then be released into the surrounding medium and are capable of providing additional activity. Since tranexamic as it is an anti-fibrinolytic, it will among other things serve to stabilize the blood clot around the graft material. Calcium has a non-negligible role in coagulation phenomena (by participating in particular in the activation of factors X and II) up to the step of transformation of the fibrinogen into fibrin monomers ready to polymerize.

The diameter of the pores predominantly present in the platelet lysate foam according to the invention is included between 0.1 and 100 μm.

This pore size is distinctive of the method for obtaining the platelet lysate foam. The drying process in supercritical CO₂ atmosphere makes it possible to obtain a foam having a diameter of the pores predominantly present included between 0.1 and 100 μm.

For example, the diameter of the pores predominantly present is included between 1 μm and 10 μm, preferably between 2 and 7 μm, and preferably between 3.2 and 4 μm. Typically the diameter of the pores predominantly present is about 3.5 μm.

The diameter of pores predominantly present may be measured by any technique known to the person skilled in the art. Typically, the mercury porosimetry test or mercury porometry test is mentioned which is an instrument for investigation of porous environments, known to the person skilled in the art.

This method consists of using pressure to force mercury (non-wetting liquid) to go inside the porous network of the material and measuring the rate of intrusion thereof in relation to the applied pressure. This method serves to determine the porosity percentage measured between 3 nm and 360 μm, and also the dimension of the pores which make up the network (“AutoPore™ IV Series, Automated Mercury Porosimeters, Micromeritics®” brochure).

Diameter of the pores predominantly present is understood to mean the diameter of the pores for which the mercury porosimeter recorded the greatest mercury intrusion rate. The diameter of the pores predominantly present is therefore measured from the volume of mercury added.

The person skilled in the art could, for example, use the AutoPore IV device from Micromeritics® (see for example Autopore IV Operator's manual, Micromeritics 2004) in order to measure the diameter of the pores predominantly present.

Advantageously, this diameter of the pores predominantly present allows colonization the material by cells and also diffusion of surrounding fluids, ions and molecules into the core of the biomaterial.

The foam according to the invention, just the same, has pores whose diameter varies from 7 nm (allowing diffusion of fluids) to 100 μm (allowing the passage of blood cells and vessels). The diameter of the pores and also the pores predominantly present are shown in FIG. 4.

According to an embodiment, the platelet lysate foam according to the invention has an average porosity included between 70% to 95%, preferably between 75% and 90%, even more preferably between 75% and 82%, again preferably between 79% and 89%, and even more preferred 77 and 89%.

Preferably, the foam has an average porosity of about 80%.

Average porosity, or porosity rate, is understood to mean the volume of the average porous network of the material corresponding to the volume not occupied by the matter making up the material. It indicates the spaces between the fibers of the network into which the fluids, molecules and later the cells could move. A porosity rate that is too low will limit the diffusion phenomena of the cells and colonization thereby of the foam and the gel corresponding to the hydrated foam.

The porosity of the foam may be measured by any technique known to the person skilled in the art. Mercury porosimetry can also be indicated for illustration.

The person skilled in the art could, for example, use the AutoPore IV device from Micromeritics® (see for example Autopore IV Operator's manual, Micromeritics 2004) in order to measure the average porosity of the foam.

The platelet lysate foam according to the present invention advantageously retains the three-dimensional arrangement of the fibrin network thereof and allows release of growth factors into the medium over time. The growth factors are in fact encased in the fibrin network (i.e. within the fibrin matrix). This network is going to allow the extended release of the growth factors.

Again advantageously, the platelet lysate foam according to the present invention is a solid foam because of the polymerization.

These platelet lysate solid foams have a net increase of their compression strength compared to platelet lysate hydrogels.

The platelet lysate foams according to the invention therefore have better mechanical properties and can be easily handled with forceps or by hand without disintegrating.

Process for Getting the Platelet Lysate Foam

The present invention also relates to a process for getting a platelet lysate foam comprising the steps:
getting a hydrogel by polymerization of a platelet lysate;
substitution of the aqueous solvent by washing;
and then drying by a drying process in a supercritical CO₂ atmosphere.

Getting a Hydrogel by Polymerization of a Platelet Lysate

The platelet lysate hydrogel is obtained by polymerization of a platelet lysate or by fibrinogen polymerization, where the platelet lysate or the fibrinogen is combined with at least one element selected from a polymerization initiator, a factor supporting polymerization, a stabilizer of coagulation, an agent with which to maintain the isotonicity and the swelling of the gel, an agent supporting the breakdown of the network, and an agent supporting the bonds in the network.

Calcium chloride (CaCl₂), thrombin and genepin are notable polymerization initiators.

Advantageously, calcium chloride will also have a gelling power.

Factor XIII, 1-ethyl-3-(3-dimethylaminopropyl) carbodiimide are notable as factors supporting polymerization.

Advantageously, 1-ethyl-3-(3-dimethylaminopropyl) carbodiimide also supports creation of bonds within the network.

The following can be noted among the stabilizers of the coagulation: tranexamic acid, which is a coagulation stabilizer by anti-fibrinolytic action, amino-caproic acid, which is a stabilizer by action against breakdown of the network of fibers, fibronectin, which is a coagulation stabilizer by adhesion of cells to the extracellular matrix.

Sodium chloride (NaCl) can be noted among the agents with which to maintain the isotonicity and the swelling of the gel.

Plasminogen can be noted among the agents supporting the breakdown of the network. This breakdown of the network occurs after the biomaterial is implanted.

N-hydroxysuccinimide can be noted among the agents supporting the bonds in the network.

Other factors such as factors serving to give elasticity to the network of fibers, factors having stimulating properties for the host cells and/or antibacterial and/or anti-inflammatory properties, factors serving to simulate host cells, factors with which to do drive precipitation of crystals and create structures close to those of natural mineralized tissues, growth factors or supplemental cytokines, coagulation factors, blood clot stabilizing factors could be combined with the platelet lysate in order to get a platelet lysate foam.
Collagen and elastin can be indicated among the factors with which to give elasticity to the network of fibers and further accentuate the biomimetic properties thereof;
Hyaluronic acid can be indicated among the factors with which to give elasticity to the network of fibers;
These factors will serve to make the final biomaterial more elastic.
Among the factors having stimulating properties for the host cells and/or antibacterial and/or anti-inflammatory properties, bioactive ions can be indicated.
The bioactive ions correspond to cations known for their biological activity, such as Sr2+, Mg2+, Cu2+, Zn2+, Ag+.
Among the factors serving to stimulate the host cells, recombinant BMP-2 can be indicated.
Factors such as recombinant BMP-2 will serve to give the biomaterial a function of stimulating bone regeneration.
Among the factors with which to drive precipitation of crystals and create structures close to those of mineralized natural tissues, calcium phosphate can be indicated.
Typically, these factors serve to precipitate calcium phosphate crystals analogous to the crystals which make up the mineral phase of natural bone.

The supplemental growth factors or cytokines are selected from members of the TGFβ (transforming growth factor β) superfamily, isoforms of the platelet origin growth factor (platelet-derived growth factor or PDGF), growth factors from the EGF family (epithelial growth factor), and VEGF (vascular endothelial growth factor).
Among the members of the TGFβ superfamily, the following can be indicated: members of the subfamily of activins such as inhibin A and inhibin B, the members of the subfamily of the Drosophila decapentaplegic (dpp) gene which include the genes coding for osseous morphogenesis, BMP4 factor and the osteogenesis factors BMP3, BMPS, BMP6, BMP7, BMP8 from the 60A subfamily. All these factors have an activity inducing cartilage and bone formation.
The following can be indicated among members the EGF family: amphiregulin (AR), TGF-α (transforming growth factor a), epigen (EPG), betacellulin (BTC), HB-EGF (heparin-binding EGF), epiregulin (EPR), neuregulin (NRG).
PDGF-AA and PDGF-BB can be indicated among the isoforms of PDGF.
The following can be indicated among the family of VEGF peptides: PIGF, VEGF-C and VEGF-B.
Among coagulation factors, thrombin can be indicated.
The following can be indicated among the clot stabilizing factors: alpha-1 antitrypsin (serine protease inhibitor), aprotinin (anti-fibrinolytic) or even amino-caproic acid (plasmin inhibitor).

According to an embodiment, the platelet lysate hydrogel is obtained by polymerization of a platelet lysate, where the platelet lysate is combined with at least one element selected from calcium chloride (CaCl₂), sodium chloride (NaCl), thrombin, amino-caproic acid, factor XIII, fibronectin, plasminogen, 1-ethyl-3-(3-dimethylaminopropyl) carbodiimide, N-hydroxysuccinimide, genipin, tranexamic acid (or 4-(methylamino)cyclohexanecarboxylic acid).

In an embodiment, the platelet lysate hydrogel is obtained from fibrinogen combined with at least one element selected from calcium chloride (CaCl₂), sodium chloride (NaCl), thrombin, amino-caproic acid, factor XIII, fibronectin, plasminogen, 1-ethyl-3-(3-dimethylaminopropyl) carbodiimide, N-hydroxysuccinimide, genipin, tranexamic acid.

According to an embodiment, the hydrogel was obtained by polymerization of a platelet lysate, where said platelet lysate is combined with calcium chloride, sodium chloride and tranexamic acid.

According to an embodiment, the platelet lysate represents between 60 and 80% by volume, the calcium chloride represents between 2 and 3% by volume, the sodium chloride represents between 20 and 30% by volume and the tranexamic acid represents between 0.1 and 0.5% by volume.

Advantageously, the resulting hydrogel serves to obtain a three-dimensional network of fibrin which has a tight mesh in which it has been shown that human mesenchymal stromal cells can proliferate and differentiate.

According to one embodiment, the polymerization time of the hydrogel is included between 10 minutes and 12 hours, preferably 15 minutes and 1 hour, and more preferably the polymerization time is about 30 minutes, where the polymerization is done at ambient temperature.

Advantageously, this polymerization time serves to obtain a quality hydrogel and the formation of the fibrous network.

The resulting platelet lysate hydrogels are used for obtaining platelet lysate foams capable of providing the same biological properties as the platelet lysate while also demonstrating superior qualities in order for sale.

Substitution of the aqueous solvent with a polar solvent by washing;

The present invention also relates to a process for getting a platelet lysate foam comprising the steps:
getting a hydrogel by polymerization of a platelet lysate;
substitution of the aqueous solvent with a polar solvent by washing;
and then drying by a drying process in a supercritical CO₂ atmosphere.

According to an embodiment, the polar solvent is a polar solvent miscible in CO₂ selected from ethanol, acetone, benzene, butane, dioxane, ethane, ethylacetoacetate, isopropanol.

Preferably, the polar solvent is acetone or ethanol.

Typically, the hydrogel is going to be soaked in a polar solvent bath in order to remove the water contained in the platelet lysate hydrogel.

For illustration, the hydrogel is soaked in the polar solvent bath for a time between 24 hours and 96 hours, preferably between 36 hours and 72 hours. According to an embodiment, the hydrogel is soaked in the organic solvent bath for about 48 hours.

After the soaking step, the hydrogel is separated from the support thereof before being placed in the closed reaction vessel of the dryer for drying with supercritical CO₂.

Drying Process in a Supercritical CO₂ Atmosphere

According to an embodiment, the step of drying with supercritical CO₂ comprises a preliminary rinsing step, where this step advantageously comprises between 1 and 5 rinses in liquid CO₂ or with supercritical CO₂.

The step of rinsing with CO₂ service to eliminate the polar solvent trapped in the hydrogel and substitute it with liquid or supercritical CO₂. Rinsing with CO₂ serves to eliminate all solvent residues and prevents shrinking of the resulting three-dimensional fibrous network. The architecture of the hydrogel is thus maintained.

Typically, the step of rinsing with supercritical CO₂ is done by circulation of supercritical CO₂ in the reaction vessel.

Typically, three steps of rinsing with liquid or supercritical CO₂ could be done.

For illustration, the steps of rinsing with liquid CO₂ are done at a temperature of 5° C. and a pressure of 40 to 50 bars, each rinsing lasts about one hour.

According to an embodiment, the supercritical atmosphere is reached by increasing the temperature beyond 39° C. and the pressure beyond 90 bars, and then holding between 10 minutes and 12 hours, preferably between 30 minutes and 10 hours, preferably between one hour and eight hours, preferentially between two hours and six hours, again preferentially between three hours and five hours, preferentially for four hours.

Holding the supercritical atmosphere for about four hours allows penetration of CO₂ into the core of the network.

Advantageously, holding the supercritical atmosphere serves to maintain the three-dimensional structure and the drying into the core of the network.

Typically, holding the supercritical atmosphere is done at a temperature of about 40° C. and a pressure of about 90 bars.

According to an embodiment, the decompression gradient is included between 1 bar/s and 20 bar/min, and is preferably 1 bar/s.

Advantageously, rapid degassing at 1 bar/s serves to yield the porous structure of the platelet lysate foam. In that way, the foam is going to be frozen. Degassing that is too rapid, i.e. faster than 1 bar/s, drives bursting of the foam. Degassing that is too slow, longer than 20 bars/min, leads to a loss of volume of the foam which is going to shrink and settle.

The step of drying with supercritical CO₂ advantageously allows maintaining the three-dimensional structure of the hydrogel during the drying operation and serves to get a platelet lysate foam having mechanical properties better than those of the initial hydrogel. Because of this process, the material is advantageously sterile without need for clean room, unlike the freeze-drying process used in the state-of-the-art.

According to an embodiment, the present invention also relates to a platelet lysate foam which could be obtained by the process from the invention.

The resulting platelet lysate foam advantageously retains the three-dimensional fibrous arrangement thereof and also the growth factors thereof with which to get biological properties identical to those from platelet lysate and the major elements thereof such as tranexamic acid, sodium, chlorine and calcium.

Use for Therapeutic Purposes and Cell Culture

The natural presence of growth factors such as TGF-β, EGF, PDGF-AB, IGF-1, VEGF and bFGF and the extended release thereof because of the fibrous three-dimensional arrangement of the platelet lysate foam according to the invention and the progressive disintegration thereof constitutes an important argument for the therapeutic use and for cell culture and therapy purposes of platelet lysate foams according to the invention.

In fact, the platelet lysate foams according to the invention are the support which allows a targeted and extended release of growth factors in situ and also supports repair or regeneration of damaged tissues.

Further, since growth factors are necessary to cell growth, proliferation and differentiation, they have a major therapeutic interest and allow the use of the material for cell therapy purposes.

VEGF (Vascular Endothelium Growth Factor) is a protein which is principally responsible for initiation of the formation of new blood vessels. It also stimulates the permeability of micro vessels and seems to be involved in the migration of monocytes/macrophages (Ehrbar M., et al., Endothelial cell proliferation and progenitor maturation by fibrin-bound VEGF variants with differential susceptibilities to local cellular activity. J Control Release Off J Control Release Soc 2005; 101(1-3):93-109). It is thus involved in neo-angiogenesis, and proliferation and migration of endothelial cells.

PDGF (platelet-derived growth factor), in interaction with the tyrosine kinase receiver, is also involved in cell growth and multiplication during angiogenesis, skin formation or even renal development (Andrae J, et al. Role of platelet-derived growth factors in physiology and medicine. Genes Dev2008; 22(10):1276-1312).

EGF (Epidermal Growth Factor) supports cell proliferation, migration and differentiation during the formation of the epithelial, cardiovascular and nervous system (Zeng F, Harris R C. Epidermal growth factor, from gene organization to bedside. Semin Cell Dev Biol 2014; 28:2-11).

TGF-β (Transforming Growth Factor) is classified as a cytokine and is mainly involved in the growth of tissues upon binding to the receptor thereof linked to the Smad pathway (hi Y, Massagué J. Mechanisms of TGF-β Signaling from Cell Membrane to the Nucleus. Cell 2003; 113(6):685-700).

IGF1 (insulin-like growth factor) allows growth in particular by stimulation of cartilage formation (Wang J, Zhou J, Bondy C A. Igf 1 promotes longitudinal bone growth by insulin-like actions augmenting chondrocyte hypertrophy. FASEB J 1999; 13:1985-90; PMID:10544181).

bFGF (basic fibroblast growth factor or FGF2) is involved in very many processes of cell proliferation, healing, regeneration and even embryogenesis (Dvorak et al. Expression and Potential Role of Fibroblast Growth Factor 2 and Its Receptors in Human Embryonic Stem Cells, Stem Cells 2005; 23:1200-1211)

Use for Cell Culture

In an embodiment, the present invention relates to the use of platelet lysate foam according to the invention for cell culture.

In fact, platelet lysate was proposed as an alternative to the use of fetal bovine serum (FBS), the most used supplement for cell culture media (Shanbhag S et al., Efficacy of Humanized Mesenchymal Stem Cell Cultures for Bone Tissue Engineering: A Systematic Review with a Focus on Platelet Derivatives. Tissue Eng Part B Rev 2017; 23(6):552-569.), because of the potential presence in the FBS of xenogenic pathogen agents (the risk of contamination by prions and viruses is not zero). In fact, regrouped human platelet lysates (hPL) do not have a risk of immune rejection or transmission of xenogenic pathogens. The culture of stem cells in platelet lysate enriched media serve both to validate the therapeutic use of these cells in people and also it was shown that generally mesenchymal stromal cells had better proliferation rates and greater metabolic activity in the presence of platelet lysate (Ma J, et al. Osteogenic capacity of human BM-MSCs, AT-MSCs and their co-cultures using HUVECs in FBS and PL supplemented media. J Tissue Eng Regen Med 2015; 9(7):779-788).

Use for Cell Therapy Purposes

The implementation of a foam which has the same biological properties as platelet lysate and which is made up of a porous three-dimensional network which supports cell invasion, proliferation and differentiation constitutes a major interest for use thereof in the cell therapy domain.

Thus, the present invention also relates to a platelet lysate foam for use thereof in a cell therapy method.

The present invention also relates to the use of a platelet lysate foam according to the invention in a cell therapy method.

The present invention also relates to a cell therapy method comprising administration, to a patient needing it, of a platelet lysate foam according to the invention.

The present invention also relates to the use of a platelet lysate foam according to the present invention for production of a medication intended for a cell therapy method.

Use for Therapeutic Purposes

Use to Support Skin Healing, Regeneration of the Dermis and Tissue Regeneration

For treating chronic skin ulcers and supporting skin healing, particles of calcium alginate (Mori M, et al. Calcium alginate particles for the combined delivery of platelet lysate and vancomycin hydrochloride in chronic skin ulcers. Int J Pharm 2014; 461(1-2):505-513), collagen gels (Lima A C, Mano J F, Concheiro A, Alvarez-Lorenzo C. Fast and mild strategy, using superhydrophobic surfaces, to produce collagen/platelet lysate gel beads for skin regeneration. Stem Cell Rev 2015; 11(1):161-179.), chitosan glutamate and sodium hyaluronate based spongy dressings (Rossi S, Faccendini A, Bonferoni M C, Ferrari F, Sandri G, Del Fante C, et al. “Sponge-like” dressings based on biopolymers for the delivery of platelet lysate to skin chronic wounds. Int J Pharm 2013; 440(2):207-215), porous silica microparticles (Fontana F, Mori M, Riva F, Mäkilä E, Liu D, Salonen J, et al. Platelet Lysate-Modified Porous Silicon Microparticles for Enhanced Cell Proliferation in Wound Healing Applications. ACS Appl Mater Interfaces 2016; 8(1):988-996) and ionic micelles of chitosan and oleic acid loaded with silver sulfadiazine (Dellera E, Bonferoni M C, Sandri G, Rossi S, Ferrari F, Del Fante C, et al. Development of chitosan oleate ionic micelles loaded with silver sulfadiazine to be associated with platelet lysate for application in wound healing. Eur J Pharm Biopharm Off J Arbeitsgemeinschaft Pharm Verfahrenstechnik EV 2014; 88(3):643-650) were proposed. These materials were designed in order to allow the absorption of platelet lysate and the progressive release of growth factors to the surface of the skin and also the proliferation of fibroblasts in the network created by the platelet lysate.

For repair of the dermis of the skin, platelet lysate solutions have been tested in direct application on rat wounds, concluding that such treatments were favorable to healing, with an effect which increased in relation to the concentration other platelet lysate solutions (Sergeeva N S, Shanskii Y D, Sviridova I K, Karalkin P A, Kirsanova V A, Akhmedova S A, et al. Analysis of Reparative Activity of Platelet Lysate: Effect on Cell Monolayer Recovery In Vitro and Skin Wound Healing In Vivo. Bull Exp Biol Med 2016; 162(1):138-145).

Other materials previously impregnated with platelet lysate were proposed in order to increase the residence time of the platelet lysate near the wound and thus increase the efficacy of the treatment. Thus, a collagen/gelatin matrix was tested in mice (Ito R, Morimoto N, Pham L H, Taira T, Kawai K, Suzuki S. Efficacy of the controlled release of concentrated platelet lysate from a collagen/gelatin scaffold for dermis-like tissue regeneration. Tissue Eng Part A 2013; 19(11-12):1398-1405) and pectin/chitosan particles (Tenci M, Rossi S, Bonferoni M C, Sandri G, Boselli C, Di Lorenzo A, et al. Particulate systems based on pectin/chitosan association for the delivery of manuka honey components and platelet lysate in chronic skin ulcers. Int J Pharm 2016; 509(1-2):59-70) were applied to rat wounds.

In the field of plastic surgery, implants of synthetic porous polyethylene (PP) were widely used for three-dimensional reconstruction of lost or severely deformed tissues. Platelet lysate is then used in combination with three-dimensional PP implants in order to reduce postoperative complications (Ozturk S, Sahin C, Tas A C, Muftuoglu T, Karagoz H. Effect of Allogeneic Platelet Lysate and Cyanoacrylate Tissue Glue on the Fibrovascularization of the Porous Polyethylene Implant. J Craniofac Surg 2016; 27(1):253-257).

The present invention therefore relates to a platelet lysate foam according to the present invention for use thereof in a method for supporting skin healing, regeneration of the dermis and tissue regeneration.

The present invention also relates to the use of a platelet lysate foam according to the invention for supporting skin healing, regeneration of the dermis and tissue regeneration.

The present invention also relates to a treatment method supporting skin healing, regeneration of the dermis and tissue regeneration comprising administration, to a patient needing it, of a platelet lysate foam according to the invention.

The present invention also relates to the use of a platelet lysate foam according to the present invention for production of a medication intended to support skin healing, regeneration of the dermis and tissue regeneration.

The term “support” is not an absolute term, and, when applied to cutaneous healing, regeneration of the dermis and tissue regeneration, it designates a procedure or a considered plan of action, even with a low probability of success, but needing to induce an overall beneficial effect such as reducing the severity of one or more symptoms or stabilizing.

Typically, “supporting skin healing, regeneration of the dermis and tissue regeneration” is understood to mean the improvement of platelet hemostasis, clot formation, clot stabilization and recruitment of inflammatory cells under the influence of the platelet lysate foam according to the invention, but also improvement of the remodeling of the extracellular matrix and good progress of the healing mechanisms.

According to an embodiment, the platelet lysate foam according to the present invention is used for use thereof in the treatment of chronic skin ulcers.

The present invention also relates to the use of a platelet lysate foam according to the invention for the treatment of chronic skin ulcers.

The present invention also relates to a treatment method for chronic skin ulcers comprising administration, to a patient needing it, of a platelet lysate foam according to the invention.

The present invention also relates to the use of a platelet lysate foam according to the present invention for production of a medication intended for the treatment of chronic skin ulcers.

Use for Supporting Osteogenesis and Bone Regeneration

Evaluations of bone formation and ectopic subcutaneous sites or directly on bone models have been conducted for about 10 years. The capacity of the platelet lysate to support differentiation of the MSC into cells of the osteoblast line is obvious and allows them to generate bone formation even on non-osseous sites (Chevallier N, Anagnostou F, Zilber S, Bodivit G, Maurin S, Barrault A, et al. Osteoblastic differentiation of human mesenchymal stem cells with platelet lysate. Biomaterials 2010; 31(2):270-278). The combination of platelet lysate with mesenchymal stromal cells is promising and requires finding an appropriate matrix. Matrices made up of collagen and fibrin were thus tested successfully on a hip prosthesis model in sheep (Dozza B, Di Bella C, Lucarelli E, Giavaresi G, Fini M, Tazzari P L, et al. Mesenchymal stem cells and platelet lysate in fibrin or collagen scaffold promote non-cemented hip prosthesis integration. J Orthop Res Off Publ Orthop Res Soc 2011; 29(6):961-968). Chakar et al. studied the osteogenic potential of platelet lysate alone, respectively with rabbit femurs and calvaria, and show that autologous platelet lysate was able to get bone regeneration (Chakar C, Naaman N, Soffer E, Cohen N, El Osta N, Petite H, et al. Bone formation with deproteinized bovine bone mineral or biphasic calcium phosphate in the presence of autologous platelet lysate: comparative investigation in rabbit. Int J Biomater 2014; 2014:367265; Chakar C, Soffer E, Cohen N, Petite H, Naaman N, Anagnostou F. Vertical bone regeneration with deproteinised bovine bone mineral or biphasic calcium phosphate in the rabbit calvarium: effect of autologous platelet lysate. J Mater Sci Mater Med 2015; 26(1):5339).

Thus, the present invention also relates to a platelet lysate foam according to the present invention for use thereof in a method for supporting osteogenesis and bone regeneration.

The present invention also relates to the use of a platelet lysate foam according to the invention for supporting osteogenesis and bone regeneration.

The present invention also relates to a method supporting osteogenesis and bone regeneration comprising administration, to a patient needing it, of a platelet lysate foam according to the invention.

The present invention also relates to the use of a platelet lysate foam according to the present invention for production of a medication intended to support osteogenesis and bone regeneration.

The term “support” is not an absolute term, and, when applied to osteogenesis and bone regeneration, it designates a procedure or a considered plan of action, even with a low probability of success, but needing to induce an overall beneficial effect such as reducing the severity of one or more symptoms or stabilizing.

Typically, “support osteogenesis and bone regeneration” is understood to mean the capacity of the platelet lysate foam to improve differentiation of the MSC into cells of the osteoblast line, by the continuous and progressive release of growth factors and cytokines and thus to generate bone formation, in order to increase the bone quantity and support mineralization thereof.

In the treatment of arthritis, intra-articular injection of autologous platelet lysates has been done in arthritic horses thus significantly improving the physical performance of the animals (Tyrnenopoulou P, Diakakis N, Karayannopoulou M, Savvas I, Koliakos G. Evaluation of intra-articular injection of autologous platelet lysate (PL) in horses with osteoarthritis of the distal interphalangeal joint. Vet Q 2016; 36(2):56-62). The authors have concluded that autologous platelet lysate injected into joints is an effective method for temporarily managing arthritis of the distal interphalangeal joint in horses involved in sports.

Thus, the present invention also relates to a platelet lysate foam according to the present invention for use thereof in treatment of arthritis.

The present invention also relates to the use of a platelet lysate foam according to the invention for the treatment of arthritis.

The present invention also relates to a treatment method for arthritis comprising administration, to a patient needing it, of a platelet lysate foam according to the invention.

The present invention also relates to the use of a platelet lysate foam according to the present invention for production of a medication intended for the treatment of arthritis.

Use for Cartilage Regeneration

In the context of regenerating cartilage, the release in situ of platelet lysate is used to support the differentiation of mesenchymal stromal cells to a chondroblastic phenotype and thus support the repair/regeneration of deficient or damaged cartilage. A hydrogel of chitosan and chondroitin sulfate capable of absorbing the platelet lysate was therefore proposed (Santo V E, Popa E G, Mano J F, Gomes M E, Reis R L. Natural assembly of platelet lysate-loaded nanocarriers into enriched 3D hydrogels for cartilage regeneration. Acta Biomater 2015; 19:56-65).

Based on the same principle, in the repair of tendons, the proposed biomaterials are intended to serve as reservoirs of impregnated biomolecules at the time of use and capable of supporting/enhancing the activity of the cells present in situ or that of cells derived from human tendons (hTDC) combined at the moment of implanting.

The materials are then patches of platelet lysates crosslinked by genipin (Costa-Almeida R, Franco A R, Pesqueira T, Oliveira M B, Babo P S, Leonor I B, et al. The effects of platelet lysate patches on the activity of tendon-derived cells. Acta Biomater 2018. doi:10.1016/j.actbio.2018.01.006), sodium alginate hydrogels and chondroitin sulfate (Sandri G, Bonferoni M C, Rossi S, Ferrari F, Mori M, Cervio M, et al. Platelet lysate embedded scaffolds for skin regeneration. Expert Opin Drug Deliv 2015; 12(4):525-545) or of photocross-linkable hydrogel composed of methacrylated chondroitin sulfate (MA-CS) enriched with iron-based magnetic nanoparticles (Silva E D, Babo P S, Costa-Almeida R, Domingues R M A, Mendes B B, Paz E, et al. Multifunctional magnetic-responsive hydrogels to engineer tendon-to-bone interface. Nanomedicine Nanotechnol Biol Med 2017. doi:10.1016/j.nano.2017.06.002).

Thus, the present invention also relates to a platelet lysate foam according to the present invention for use thereof in a method for supporting cartilage regeneration.

The present invention also relates to the use of a platelet lysate foam according to the invention for supporting cartilage regeneration.

The present invention also relates to a treatment method supporting cartilage regeneration comprising administration, to a patient needing it, of a platelet lysate foam according to the invention.

The present invention also relates to the use of a platelet lysate foam according to the present invention for production of a medication intended for cartilage regeneration.

The term “support” is not an absolute term, and, when applied to cartilage regeneration, it designates a procedure or a considered plan of action, even with a low probability of success, but needing to induce an overall beneficial effect such as reducing the severity of one or more symptoms or stabilizing.

Typically, “supporting cartilage regeneration” is understood to mean the capacity of the platelet lysate foam to support the differentiation of mesenchymal stromal cells to a chondroblastic phenotype and thus support the repair/regeneration of deficient or damaged cartilage.

Use for Treatment of Corneal Lesions Such as Chronic Lesions of the Cornea

In the treatment of chronic wounds of the eye, the arrangements must allow increasing the pre-cornmeal residence time of growth factors contained in the reduced platelet lysate because of the significant drainage from the lacrimal flow triggered by lacrimation.

Thermal sensitive and mucoadhesive collyria obtained based on sodium chondroitin sulfate (CS) and hydroxypropyl methylcellulose (HPMC) (Sandri G, Bonferoni M C, Rossi S, Ferrari F, Mori M, Del Fante C, et al. Thermosensitive eyedrops containing platelet lysate for the treatment of corneal ulcers. Int J Pharm 2012; 426(1-2):1-6), chitosan or polyacrylic acid supports (Sandri G, Bonferoni M C, Rossi S, Ferrari F, Mori M, Del Fante C, et al. Platelet lysate formulations based on mucoadhesive polymers for the treatment of corneal lesions. J Pharm Pharmacol 2011; 63(2):189-198) chondroitin sulfate intended to support the maintenance of platelet lysate instilled in the eye and to thereby improve the treatment of corneal lesions (Sandri G, Bonferoni M C, Rossi S, Delfino A, Riva F, Icaro Cornaglia A, et al. Platelet lysate and chondroitin sulfate loaded contact lenses to heal corneal lesions. Int J Pharm 2016; 509(1-2):188-196).

Thus, the present invention also relates to a platelet lysate foam according to the present invention for use thereof in treatment of corneal lesions, such as chronic corneal lesions.

The present invention also relates to the use of a platelet lysate foam according to the invention for the treatment of corneal lesions, such as chronic corneal lesions.

The present invention also relates to a treatment method for corneal lesions, such as chronic corneal lesions, comprising administration, to a patient needing it, of a platelet lysate foam according to the invention.

The present invention also relates to the use of a platelet lysate foam according to the present invention for the production of a medication intended for the treatment of corneal lesions, such as chronic corneal lesions.

The term “treatment” is not an absolute term, and, when applied to treatment of corneal lesions, it designates a procedure or a considered plan of action, even with a low probability of success, but needing to induce an overall beneficial effect such as delaying the appearance of the pathology or reducing the severity of one or more symptoms or stabilizing.

Typically, the treatment of a corneal lesion is based on the capacity of the platelet lysate foam to keep the instilled platelet lysate in the eye and increase the pericorneal persistence time of growth factors contained in the platelet lysate because of the extended release.

Use in the Treatment of Neurodegenerative Disorders Such as Parkinson's Disease

Parkinson's disease, with its elevated morbidity, was recently studied in cell models treated by exposure to regrouped/pooled human platelet lysates (hPL). The results confirmed that such therapies could be used for preventing in vivo neuron loss because the platelet lysate has protective properties against cell death pathways and some oxidative stress inducers (Gouel F, Do Van B, Chou M-L, Jonneaux A, Moreau C, Bordet R, et al. The protective effect of human platelet lysate in models of neurodegenerative disease: involvement of the Akt and MEK pathways. J Tissue Eng Regen Med 2017; 11(11):3236-3240).

Also, a nasal spray containing platelet lysates was tested with encouraging results in mice with Parkinson's disease (Chou M-L, Wu J-W, Gouel F, Jonneaux A, Timmerman K, Renn T-Y, et al. Tailor-made purified human platelet lysate concentrated in neurotrophins for treatment of Parkinson's disease. Biomaterials 2017; 142:77-89).

Thus, the present invention also relates to a platelet lysate foam according to the present invention for use thereof in treatment of neurodegenerative disorders such as Parkinson's disease.

The present invention also relates to the use of a platelet lysate foam according to the invention for the treatment of neurodegenerative disorders such as Parkinson's disease.

The present invention also relates to a treatment method for neurodegenerative disorders such as Parkinson's disease comprising administration, to a patient needing it, of a platelet lysate foam according to the invention.

The present invention also relates to the use of a platelet lysate foam according to the present invention for the production of a medication intended for the treatment of neurodegenerative disorders such as Parkinson's disease.

The term “treatment” is not an absolute term, and, when applied to treatment of neurodegenerative disorders such as Parkinson's disease, it designates a procedure or a considered plan of action, even with a low probability of success, but needing to induce an overall beneficial effect such as delaying the appearance of the pathology or reducing the severity of one or more symptoms or stabilizing.

Typically, the treatment of Parkinson's disease is based on the capacity of the platelet lysate foam to prevent and/or reduce in vivo neuron loss in order to reduce the progression of Parkinson's disease and side effects thereof.

Use in the Treatment of the Effects of a CVA

Care for the side effects of severe illnesses such as cerebrovascular accidents may also be considered in presence of regrouped/pooled human platelet lysates (hPL) or pools of platelet lysates. Ischemic CVA models are common in rats for evaluating neurological deficits or motor functions after occlusion of blood vessels. Whether it is used to cultivate mesenchymal stromal cells before injection or directly injected into the ischemic sites, platelet lysate shows favorable results on post-attack neuromotor functions (Yamauchi T, Saito H, Ito M, Shichinohe H, Houkin K, Kuroda S. Platelet lysate and granulocyte-colony stimulating factor serve safe and accelerated expansion of human bone marrow stromal cells for stroke therapy. Transl Stroke Res 2014; 5(6):701-710).

Thus, the present invention also relates to a platelet lysate foam according to the present invention for use thereof for supporting neuromotor functions following a cerebrovascular accident (CVA).

The present invention also relates to the use of a platelet lysate foam according to the invention for supporting neuromotor functions following a cerebrovascular accident (CVA).

The present invention also relates to a treatment method supporting neuromotor functions following a cerebrovascular accident (CVA) comprising administration, to a patient needing it, of a platelet lysate foam according to the invention.

The present invention also relates to the use of a platelet lysate foam according to the present invention for production of a medication intended to support neuromotor functions following a cerebrovascular accident (CVA).

Use in the Periodontal Tissue Regeneration

Recent data concerning periodontal tissue regeneration has been obtained by Babo et al. who studied the interest of stabilization at contact of the dental root with proteins contained in platelet lysate, and to show that this supported the regeneration of rat periodontal tissues, in particular the tooth socket and the cement, the two tissues mineralized by periodontitis (Babo P S, Cai X, Plachokova A S, Reis R L, Jansen J, Gomes M E, et al. Evaluation of a platelet lysate bilayered system for periodontal regeneration in a rat intrabony three-wall periodontal defect. J Tissue Eng Regen Med 2017. doi:10.1002/term.2535; Babo P S, Cai X, Plachokova A S, Reis R L, Jansen J A, Gomes M E, et al. The Role of a Platelet Lysate-Based Compartmentalized System as a Carrier of Cells and Platelet-Origin Cytokines for Periodontal Tissue Regeneration. Tissue Eng Part A 2016; 22(19-20):1164-1175)

Thus, the present invention also relates to a platelet lysate foam according to the present invention for use thereof in a method for supporting periodontal tissue regeneration.

The present invention also relates to the use of a platelet lysate foam according to the invention for supporting periodontal tissue regeneration.

The present invention also relates to a treatment method supporting periodontal tissue regeneration comprising administration, to a patient needing it, of a platelet lysate foam according to the invention.

The present invention also relates to the use of a platelet lysate foam according to the present invention for production of a medication intended to support periodontal tissue regeneration.

The term “support” is not an absolute term, and, when applied to periodontal tissue regeneration, it designates a procedure or a considered plan of action, even with a low probability of success, but needing to induce an overall beneficial effect such as reducing the severity of one or more symptoms or stabilizing.

Typically, “periodontal tissue regeneration” is understood to mean the capacity of the platelet lysate foam to stabilize on contact with the dental root proteins contained in the platelet lysate and in that way to increase the quantity and density of periodontal tissues and more specifically to give the periodontal area an original structure based on the presence of cement at the surface of the tooth, tooth socket bone and desmodontal ligament between the two.

Use for Alopecia Treatment

It was shown that the lysate was capable of activating anagenic pathways supporting hair growth (Dastan M, Najafzadeh N, Abedelahi A, Sarvi M, Niapour A. Human platelet lysate versus minoxidil stimulates hair growth by activating anagen promoting signaling pathways. Biomed Pharmacother Biomedecine Pharmacother 2016; 84:979-986).

Thus, the present invention also relates to a platelet lysate foam according to the present invention for use thereof in treatment of alopecia.

The present invention also relates to the use of a platelet lysate foam according to the invention for the treatment of alopecia.

The present invention also relates to a treatment method for alopecia comprising administration, to a patient needing it, of a platelet lysate foam according to the invention.

The present invention also relates to the use of a platelet lysate foam according to the present invention for production of a medication intended for the treatment of alopecia.

### BRIEF DESCRIPTION OF THE DRAWINGS

Other characteristics, details and advantages the invention will appear upon reading the following detailed description, and analyzing the attached drawings, on which:

FIG. 1 shows the compression strength of the platelet lysate foams according to the invention (“Foams”), in comparison with the initial hydrogels (“Hydrogels”) (n=12);

FIG. 2 shows the breakdown kinetics in aqueous medium of the platelet lysate foam according to the invention;

FIG. 3 shows the release of VEGF growth factor (in pg/mL) over time (in days) as a function of the various forms (platelet lysate foam according to the invention, platelet lysate hydrogel and control liquid).

### EXAMPLES

### Example 1

### Getting Hydrogel from Platelet Lysate

Platelet lysate hydrogels were obtained from platelet lysate combined with various elements in liquid form according to the proportions as summarized in Table 1 below:

TABLE 1

| Constituents | Proportion (%) |
|---|---|
| Platelet Lysate | between 60 and 80% |
| CaCl₂ | between 2 and 3% |
| NaCl | between 20 and 30% |
| Tranexamic acid | between 0.1 and 0.5% |

The resulting hydrogels have optimal fibrous and porous structures, in particular for promoting cell proliferation, migration and differentiation.

Advantageously these platelet lysate hydrogels are used for obtaining platelet lysate foams capable of providing the same properties as the platelet lysate while also demonstrating superior qualities for sale. The use of platelet lysate foams is easier and may be suitable to all pathologies treated by tissue or cell engineering.

### Example 2

### Process for Getting the Platelet Lysate Foam

The platelet lysate hydrogel is then dried in the reaction vessel of a supercritical CO₂ dryer. This type of reaction vessel advantageously allows maintaining the three-dimensional structure of a hydrogel during the drying operation.

In order to extract the water contained in the platelet lysate hydrogel, it is soaked for 48 hours in an acetone bath and then separated from the support thereof before being placed in the closed reaction vessel of the dryer. Preferably, the hydrogel is soaked in a glass or metal container.

The temperature of the reaction vessel chamber is lowered to a temperature below 10° C. in order to allow liquid CO₂ to enter. The reaction vessel is filled with the liquid CO₂ until immersing the samples and then the assembly is left to soak for 45 minutes in order to allow the liquid CO₂ to penetrate the porous network of the gel. Rinsing is then done by emptying the CO₂ present in the chamber and then letting in the same new quantity of liquid. There soaking/rinsing operation is repeated three times. After the cycles, the reservoir is again filled halfway, the reaction vessel closed and then the temperature is progressively raised to 40° C. and the pressure up to 90 bar. Since the reaction vessel was closed, when the temperature increases, the pressure inside the reaction vessel increases. The supercritical state, which corresponds to the fourth state of matter, is reached when the temperature is over 31° C. and the pressure over 74 bars. The reaction vessel was held at that temperature and that pressure for four hours and then rapidly degassed and depressurized over 90 seconds.

All of the acetone present in the hydrogel was replaced by liquid CO₂ during the soaking/rinsing phases, and then when increasing the temperature and pressure, any trace of solvent is eliminated, the fiber network is then dry and the dry gel has a porous, fibrous foam form.

As shown in the following examples, the resulting platelet lysate foam advantageously keeps the three-dimensional fibrous arrangement thereof (example 3) and the major elements such as sodium, chlorine, phosphorus, sulfur and calcium (example 4).

The platelet lysate foam additionally has better mechanical properties than those of the initial hydrogel (example 5). The resulting foam is a dry material capable of being easily stored and rehydrated (example 6), which supports the rapid penetration of biological fluids and cells but also the cell activity by releasing growth factors and other proteins (example 7).

### Example 3

### Characterization of the Microstructure

The fiber network of the platelet lysate foam was observed under environmental scanning electron microscope with metallization before and after drying with supercritical CO₂.

The process serves to get a fibrous network such as a three-dimensional matrix. Advantageously, the fibrin network retains the three-dimensional fibrous arrangement thereof.

The mesh of the fibrous network is larger after drying which allows checking the porosity. It is thus possible to change the diffusion phenomena inside the porous material by modifying the average porosity and average diameter of the pores predominantly present in the three-dimensional network.

Thus, in that way it is possible to change the penetration of fluids and cells (and also the growth factor release kinetics).

These two parameters change the growth factor release kinetics and of anything which may have been incorporated in the foam. This does not change the quantity released but the speed at which the growth factors are going to be released and likewise, the duration of action of the foam.

In general, the speed of release increases when the average porosity increases and when the average size of the pores increases.

The porosity of the platelet lysate foam was quantified and the porous network was characterized.

The method used is mercury porosimetry (device: Autopore III, Micromeritics). The method consists of having the mercury enter into the pores of the platelet lysate foam under increasing pressure. The platelet lysate foam sample is going to be weighed in a conductance cell before and after filling with mercury. An analysis of the mercury pressure differential is going to be done in order to quantify the porosity and characterize the porous network.

Advantageously, the platelet lysate foams according to the invention have an average porosity of around 80%. Advantageously, an average porosity of around 80% allows fluids, molecules, ions and cells to get in between the fibers of the network and thus support penetration thereof.

The diameter of the pores predominantly present in the platelet lysate foam is 3.5 μm. Advantageously, this diameter of pores predominantly present allows fluids, ions, molecules and cells in the environment to enter all the way into the core of the network.

It is observed that a minority of the pores have an average diameter included between 10 and 11 μm, some pores have a diameter included between 6.5 and 8 μm, and pores have an average diameter included between 0.4 and 2 μm.

### Example 4

### Characterization of the Mechanical Properties

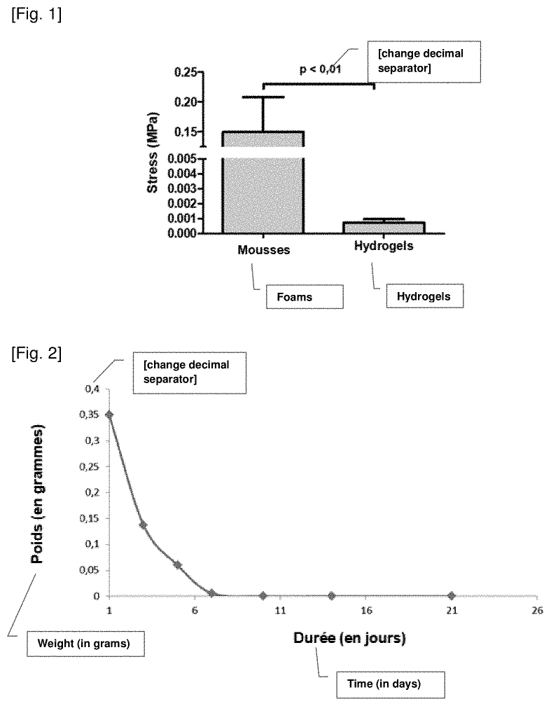

TAX T2 compression tests were done in order to characterize the mechanical properties of the platelet lysate foams dried with supercritical CO₂. These mechanical properties were compared to those of the initial hydrogels (“hydrogels” in FIG. 1).

The following were the conditions:
Loading speed: 2 mm/min;
Analysis of the behavior up to 60% compression;
Device: TA.XT Plus Texture Analyzer.

The dry networks have a distinct increase of their compression strength compared to the initial hydrogels (n=4; p<0.01).

The platelet lysate foams according to the invention therefore have better mechanical properties than those of the initial hydrogel. These foams can thus be easily handled with forceps or by hand without disintegrating as the hydrogel does.

### Example 5

### Determination of the Rehydration Rate after Drying

The rehydration rate after drying the platelet lysate foams according to the invention was determined. The method used is the weighing method.

The following are the conditions:
The samples were soaked in 1200 μL of water at 25° C. for 48 hours;
The rehydration rate is calculated using the formula:

$$rehydraton rate = \frac{( wet weight - dry weight )}{dry weight} \times 100 \qquad [ Math . 1 ]$$

The average rehydration rate calculated is 804.9%.

The platelet lysate foam therefore has a significant rehydration rate. Also advantageously, and in the absence of water, the platelet lysate foam has a favorable storage for sale thereof. In fact, and in the absence of water, the dry material does not break down over time.

### Example 6

### Breakdown and Extended Release Kinetics

The breakdown kinetics in aqueous medium and release of a growth factor included in the platelet lysate foam were evaluated.

Breakdown kinetics in aqueous medium

The method used is the weighing method.

The following are the conditions:
Samples soaked in 20 mL of water at 25° C.;
Tracking of the breakdown by weighing of the material.

As shown in FIG. 2, the platelet lysate foam according to the invention disintegrates at the end of five days. The growth factors therefore have an extended release and are not released immediately as is the case with the platelet lysate liquid.

Release of a Growth Factor, VEGF

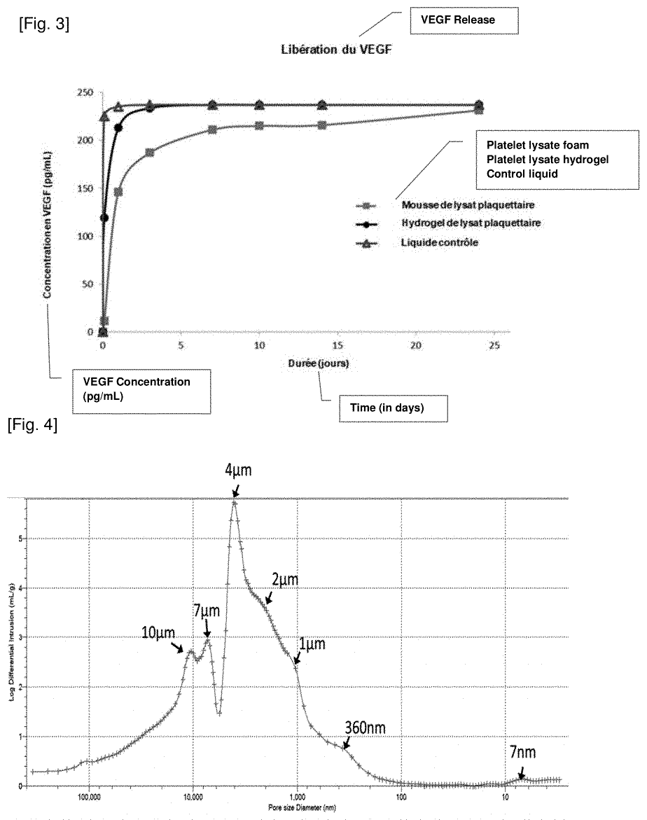

The VEGF was assayed in order to evaluate the release thereof. The method used is that of the Human VEGF Pre-Coated ELISA Kit test from Biogems. The release of the VEGF by the platelet lysate foam according to the invention was compared with the release kinetics of the platelet lysate hydrogel. A liquid was used as control, as is shown in FIG. 3.

The platelet lysate hydrogel was prepared by the process described in example 1 and the platelet lysate foam was prepared by the process described in example 2.

The measurement of the absorbance was done at 450 nm.

As shown in FIG. 3, the VEGF is released progressively over five days until reaching the maximum concentration thereof. The extended release of VEGF continued for 25 days.

In that way, and advantageously, the platelet lysate foam according to the present invention initially composed based on platelet lysate rich in growth factors, released VEGF over time. That shows that the growth factors are encased in the fibrin network and are accessible to the cells.

This release is extended and in similar quantity to that of the platelet lysate hydrogel, confirming there is no loss of protein material during the drying process.

Therefore advantageously, the platelet lysate foams according to the invention may be used in many biological applications such as regeneration and repair of damaged tissues.

In fact, the natural presence of growth factors and cytokines such as VEGF, PDGF, EGF and TGFβ which are released during implantation in the medium of the platelet lysate foam according to the invention, thus contributing to the growth of tissues and the development of organs, constitutes an important argument for the biomedical use of platelet lysate foams according to the invention.

Beyond the advantage thereof from extended release compared to the platelet lysate hydrogels, the platelet lysate foams according to the invention are further easier to handle and have improved storage and mechanical properties.

1. A platelet lysate foam characterized in that it comprises TGF-β, EGF, PDGF-AB, IGF-1, VEGF and bFGF within a polymerized fibrin matrix.

2. The platelet lysate foam according to claim 1, further comprising calcium and/or tranexamic acid.

3. The platelet lysate foam according to claim 1, characterized in that said foam has a porosity of between 70% and 95%.

4. A process for making a platelet lysate foam comprising the steps of:
providing a hydrogel by polymerization of a platelet lysate;
substituting aqueous solvent with a polar solvent by washing;
and then drying by a drying process in a supercritical CO₂ atmosphere.

5. The process according to claim 4 characterized in that the hydrogel is obtained by polymerization of a platelet lysate, where said platelet lysate is combined with a polymerization initiator, with an agent with which to maintain the isotonicity and the swelling of the gel, and with a coagulation stabilizer.

6. The platelet lysate foam obtained by the process of claim 4.

7. A method of cell culture, comprising p1 culturing cells in the platelet lysate foam according to claim 1.

8. A method of supporting skin healing and regeneration of the dermis, for supporting osteogenesis and bone regeneration, and/or for tissue regeneration and/or cell therapy in a patient in need thereof, comprising
administering to the patient a therapeutically effective amount of the platelet lysate foam of claim 1.

9. A method of treating corneal disorders in a patient in need thereof, comprising
administering to the patient a therapeutically effective amount of the platelet lysate foam of claim 1.

10. A method of supporting osteogenesis and bone regeneration, or for supporting periodontal tissue regeneration in a patient in need thereof, comprising
administering to the patient a therapeutically effective amount of the platelet lysate foam of claim 1.

11. The platelet lysate foam according to claim 3, wherein the foam has a porosity of about 80%.

12. The platelet lysate foam according to claim 5, wherein the polymerization initiator is calcium chloride (CaCl₂), thrombin, and/or genipin, the agent to maintain isotonicity and swelling is sodium chloride (NaCl), and the coagulation stabilizer is tranexamic acid, amino-caproic acid and/or fibronectin.
